(19) United States
(12) Patent Application Publication
(10) Pub. No.: US 2026/0263121 A1
(43) Pub. Date: Sep. 10, 2026

(54) Spinal Plate and Methods of Implantation

(71) Applicant: Alphatec Spine, Inc., Carlsbad, CA (US)

(72) Inventors: Edwin Huh, Irvine, CA (US); Michael Parks, San Diego, CA (US); Reid Berke, Encinitas, CA (US); David Morton, San Diego, CA (US); Mike Aleali, Laguna Niguel, CA (US)

(73) Assignee: Alphatec Spine, Inc., Carlsbad, CA (US)

(21) Appl. No.: 19/072,772

(22) Filed: Mar. 6, 2025

(51) Int. Cl. A61B 17/70; A61B 17/86

(52) U.S. Cl. CPC A61B 17/7059; A61B 17/86

(57) ABSTRACT

Disclosed herein are spinal plates having a plate body with an anterior surface and a posterior surface, a plurality of screw holes extending from the anterior surface through the plate body to the posterior surface, and one or more projections extending from the posterior surface, the one or more projections configured to releasably engage an interbody and allow the spinal plate to pivot relative to the interbody.

### BACKGROUND OF THE INVENTION

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease, and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy, correction, and implantable prosthetics. Implants such as bone fasteners, plates, connectors and vertebral rods are often used to provide stability to a treated region. These implants can redirect stresses away from a damaged or defective region while healing takes place to restore proper alignment and generally support the vertebral members. This disclosure describes an improvement upon these technologies.

### SUMMARY OF THE INVENTION

The present disclosure relates to various embodiments of spinal or fixation plates, various embodiments of interbodies or cages or spacers, and combinations of the two types of embodiments particular when used with bone screws, inserters, and various embodiments of tamps.

According to some embodiments, a spinal plate includes a plate body having a thickness between an anterior surface and a posterior surface, a plurality of screw holes extending from the anterior surface to the posterior surface; and one or more projections extending from the posterior surface, the projection(s) positioned and sized so as to be releasably received by a bore in an interbody device. In some embodiments, the projection(s) allows the plate body to pivot relative to the interbody device while the projection is received into the bore.

According to some embodiments, an interbody includes an anterior face and one or more bore(s) therein sized to receive projection(s) of a spinal plate as described herein.

According to some embodiments, an interbody/plate system includes, any spinal plate as described herein, any interbody as described herein, and a plurality of bone screws to be inserted through the screw holes of the interbody and advanced into respective vertebral bodies. Some embodiments, also include an inserter configured to engage a bore in the interbody so as to secure the interbody to the inserter and secure the spinal plate between a distal portion of the inserter and the interbody so that the interbody/inserter combination may be advanced to a desired surgical site (e.g., a damaged or diseased disc space) so that the interbody may be inserted into the disc space and the spinal plate positioned against a superior and an inferior vertebral body. Some embodiments further include a tamp that can be used to adjust the position of the interbody in the disc space without requiring the removal of the spinal plate.

According to some embodiments, a method of implanting any of the systems described herein includes press fitting any embodiment of a spinal plate as described herein to any embodiment of an interbody as described herein to form a spinal plate/interbody combination. In some embodiments, press fitting the spinal plate to the interbody comprises inserting projection(s) of the spinal plate into bore(s) in the interbody, which, when press fit together, allows the spinal plate to pivot relative to the interbody. Some methods further include securing a distal end of an inserter to the interbody thereby holding the spinal plate between the interbody and the inserter. Some methods further include advancing the spinal plate/interbody combination toward a surgical site and inserting the interbody into an intervertebral disc space. Some embodiments further include securing the spinal plate to a vertebral body above, cranial to, or superior to the intervertebral disc space and to a vertebral body below, caudal to, or inferior to the intervertebral disc space by advancing each one of the plurality of bone screws through the plurality of screw holes, respectively.

### BRIEF DESCRIPTION OF DRAWINGS

The embodiments set forth in the drawings are illustrative and exemplary in nature and not intended to limit the subject matter defined by the claims. The following detailed description of the illustrative embodiments can be better understood when read in conjunction with the following drawings wherein like structure is indicated with like reference numerals and in which:

FIGS. 1A-1D are views from various perspectives of one embodiment a spinal plate according to the present disclosure;

FIGS. 2A-2B are views from various perspectives of another embodiment according to the present disclosure;

FIGS. 3A-3B are views from various perspectives of another embodiment according to the present disclosure;

FIGS. 4A-4B are views from various perspectives of another embodiment according to the present disclosure;

FIGS. 5A-5B are views from various perspectives of another embodiment according to the present disclosure;

FIGS. 6A-6B are views from various perspectives of another embodiment according to the present disclosure;

FIG. 7 is a perspective view of an embodiment of an interbody according to the present disclosure;

FIGS. 8A-8E are views from various perspectives of the embodiment of FIGS. 1A-1D in combination with the interbody of FIG. 7 according to the present disclosure; and

FIGS. 9A and 9B are views of the spinal plate of FIGS. 1A-1D combined with the interbody of FIG. 7 as well as an embodiment of a tamp according to the present disclosure;

FIGS. 10A and 10B are views of the spinal plate of FIGS. 1A-1D combined with the interbody of FIG. 7 as well as another embodiment of a tamp according to the present disclosure;

FIG. 11 is a perspective of the combination of FIGS. 8A-8E and a plurality of bone screws according to the present disclosure; and

FIG. 12 is a flow diagram of an embodiment of a method according to the present disclosure.

### DETAILED DESCRIPTION OF THE INVENTION

The present disclosure relates to various fixation or spinal plates that can be used in conjunction with an interbody device (also known as a cage or spacer). In particular, the spinal plates of this disclosure achieve an aligned configuration with the interbody without actually being secured to the interbody so that the interbody and spinal plate may be adjusted or positioned independently of each other.

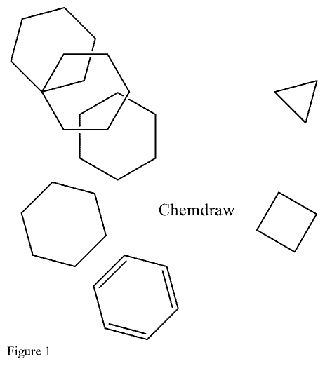

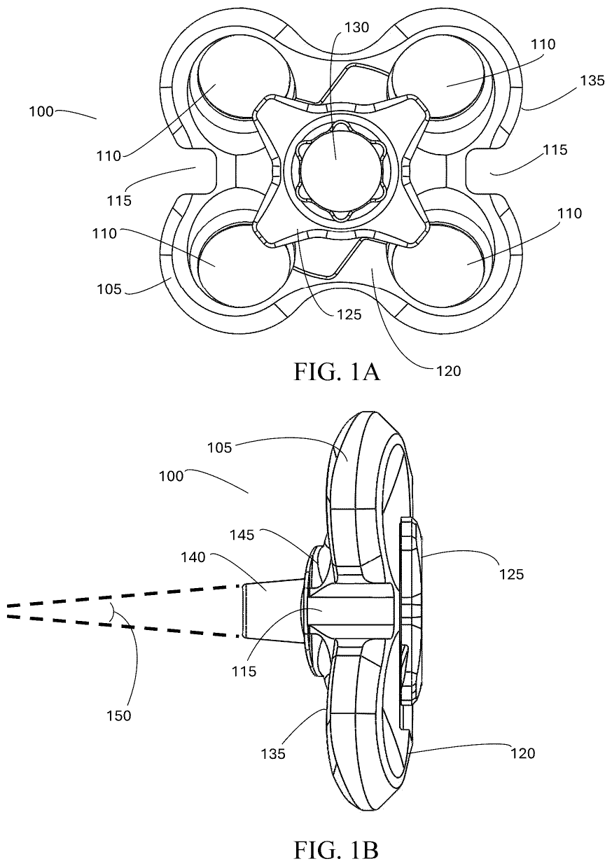

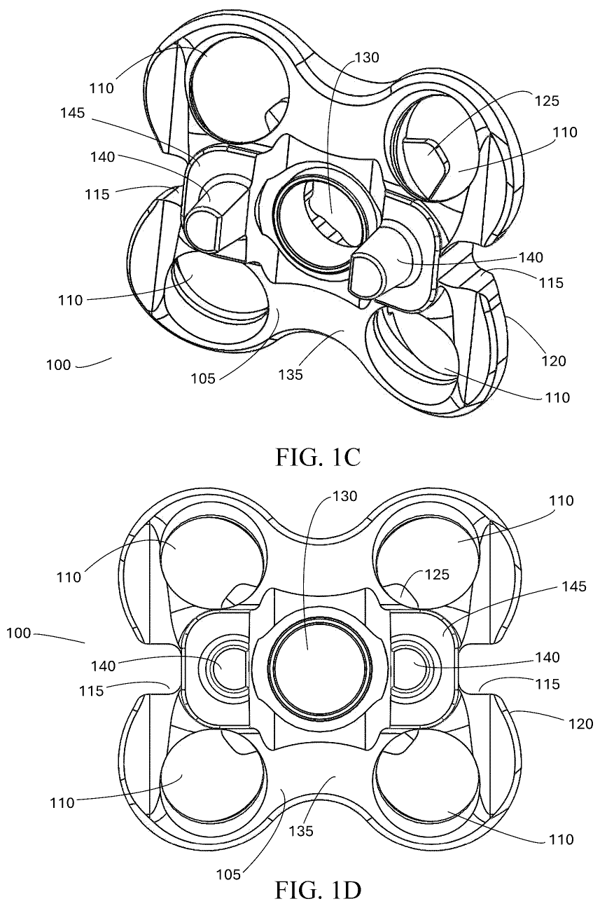

FIG. 1A illustrates one embodiment of a spinal plate 100 that includes a plate body 105. Plate body 105 has a thickness sufficient to make the spinal plate sufficiently rigid to immobilize two adjacent vertebral bodies. Plate body 105 includes a plurality of screw holes 110. In this embodiment, there are four screws holes positioned so that two holes are positioned on a cephalad portion of the plate body 105 with two holes positioned on a caudal portion of the plate body 105. Located between the cephalad and caudal portions of the plate body 105 are a pair of cutouts 115 on each lateral side of the plate body 105. The function of cutouts 115 is discussed in greater detail below.

FIG. 1A illustrates an anterior surface 120 of plate body 105 to which is secured a rotating locking mechanism 125 configured to allow respective bone screws to be inserted into each one of screw holes 110 when locking mechanism 125 is rotated, in this embodiment, by about 45°. As illustrated, locking mechanism 125 is positioned in a “closed” position meaning that a series of extensions of locking mechanism 125 extend at least partially over each respective screw hole 110. A person of skill in the art will recognize that other amounts of rotation could achieve equally favorable results. In some embodiments, no rotating locking mechanism is included in favor of a different type of locking mechanism (passive or active) or simply in favor of a slimmer plate design. At the center of locking mechanism 125 is a central hole 130 that extends from anterior surface 120 through plate body 105 to a posterior surface 135.

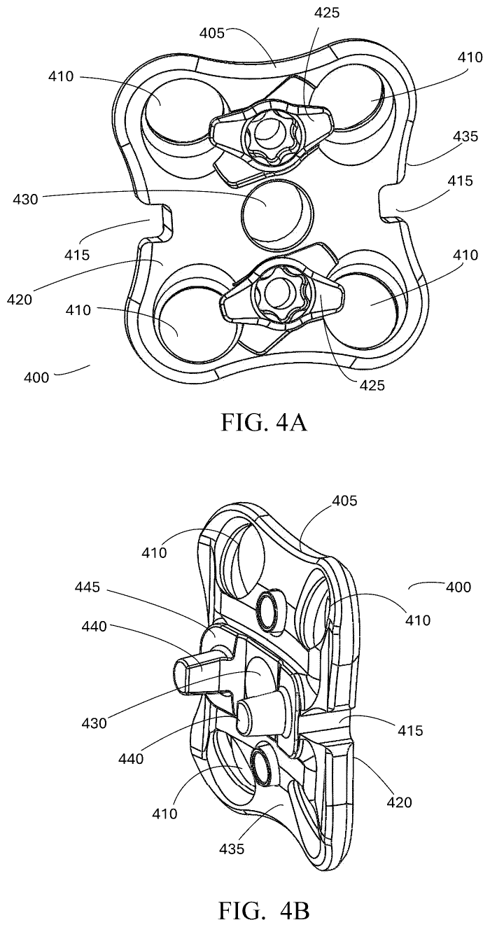

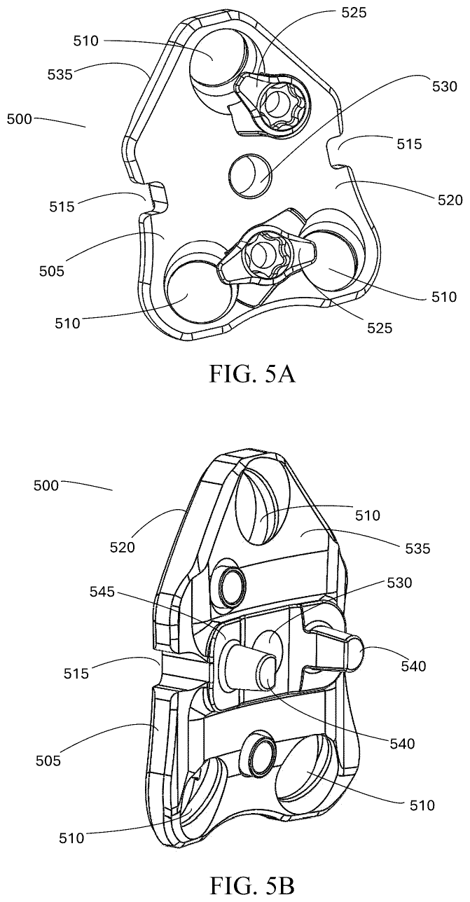

FIG. 1B is a side view of spinal plate 100 illustrating the presence of a pair of extensions 140 (best viewed in FIG. 1C) that extend from a shelf 145 located on posterior surface 135 in a posterior direction relative to plate body 105. Each extension 140 has a frustoconical shape defined by an angle 150. Each extension 140 is designed to engage with a bore or through hole on an interbody (illustrated in FIGS. 4A-4D and 5). The shape of extension 140 may be chosen to achieve a friction fit or press fit with the bore of the interbody, a friction fit that, in some embodiments, allows for a limited amount of angulation of spinal plate 100 relative to the interbody. In some embodiments the achieved friction fit or press fit may require a certain amount of force to separate the spinal plate from an interbody.

Angle 150 may be any number of suitable values that can be achieve a friction fit. In some embodiments, 150 is any value within a range of about 2 degrees to about 30 degrees, about 3 degrees to about 20 degrees, about 4 degrees to about 15 degrees, about 5 degrees to about 12 degrees, about 7 degrees to about 14 degrees, or about 9 degrees to about 16 degrees. In some embodiments, the angle is about 6 degrees, about 8 degrees, about 10 degrees, about 12 degrees, about 14 degrees, about 16 degrees, or about 18 degrees.

FIG. 1C illustrates a rear or posterior perspective view of spinal plate 100 in which both extensions 140 are visible. Some embodiments may include only a single extension 140 though having two extensions 140 may provide greater stability between spinal plate 100 and an interbody. Some embodiments may include at least three extensions. Central hole 130 is positioned directly between extensions 140 though such a configuration is not a requirement. The relative positioning of central hole 130 and extensions 140 is determined based on the relative positions of corresponding through holes or bores in the interbody. FIG. 1D similarly illustrates the symmetrical nature of these features in this embodiment.

The length of extensions 140 and/or the height of shelf 145 can be adjusted to achieve a desired positioning of an interbody within an intervertebral disc space. In some embodiments, the length and/or height is selected to achieve an interbody positioning in the disc space that is proud, flush, or sub-flush relative to one or both anterior surfaces of the adjacent vertebral bodies. In some embodiments, height of shelf 145 and the length, size, and shape of extensions 140 are selected to allow for an interbody to be positioned proud relative to one or both anterior faces of the adjacent vertebral bodies while also allowing the interbody to be advanced further into the disc space while keeping the extensions in the bores of the interbody.

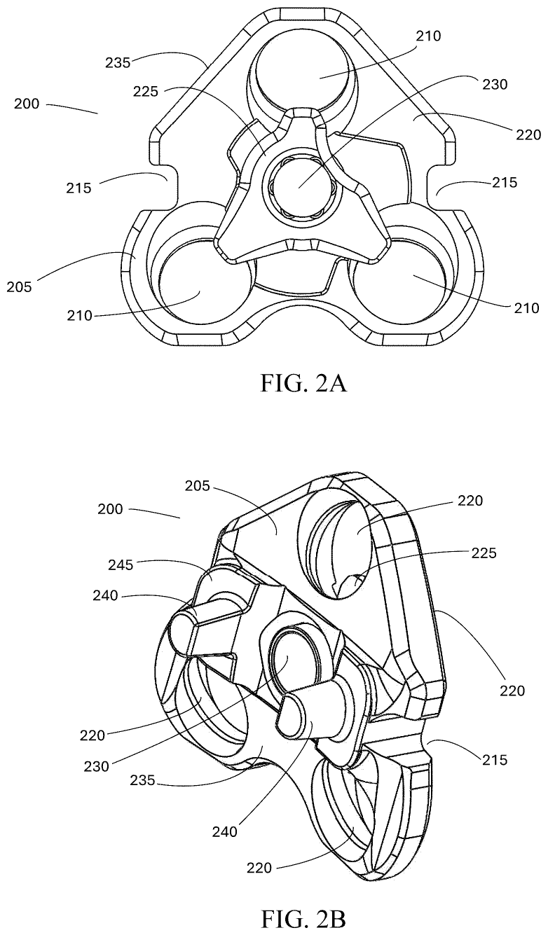

FIG. 2A illustrates another embodiment of a spinal plate 200 having a plate body 205 with an anterior surface 220, a posterior surface 235, three screw holes 210 extending from anterior surface 220 through plate body 205 to posterior surface 235. One screw hole 210 is positioned in a cephalad portion of plate body 205, and two screw holes 210 are positioned in a caudal portion of plate body 205. Located between the cephalad and caudal portions of the plate body 205 are a pair of cutouts 215 on each lateral side of the plate body 205.

Plate body 205 includes a rotating locking mechanism 225 configured to allow respective bone screws to be inserted into each one of screw holes 210 when locking mechanism 225 is rotated. At the center of locking mechanism 225 is a through hole 230 that extends from anterior surface 220 through plate body 205 to a posterior surface 235.

FIG. 2B is a rear or posterior perspective view of spinal plate 200 illustrating a pair of extensions 240 that extend from a shelf 245 on posterior surface 235 in a posterior direction relative to plate body 205. Each extension 240 has a frustoconical shape. The shape of extension 240 may be chosen to achieve a friction fit with a bore or through hole of an interbody, a friction fit that, in some embodiments, allows for a limited amount of angulation of spinal plate 200 relative to the interbody. Some embodiments may include only a single extension 240 though having two extensions 240 may provide greater stability between spinal plate 200 and an interbody. Through hole 230 is positioned directly between extensions 240 though such a configuration is not a requirement. The relative positioning of through hole 230 and extensions 240 is determined based on the relative positions of corresponding through holes or bores in the interbody.

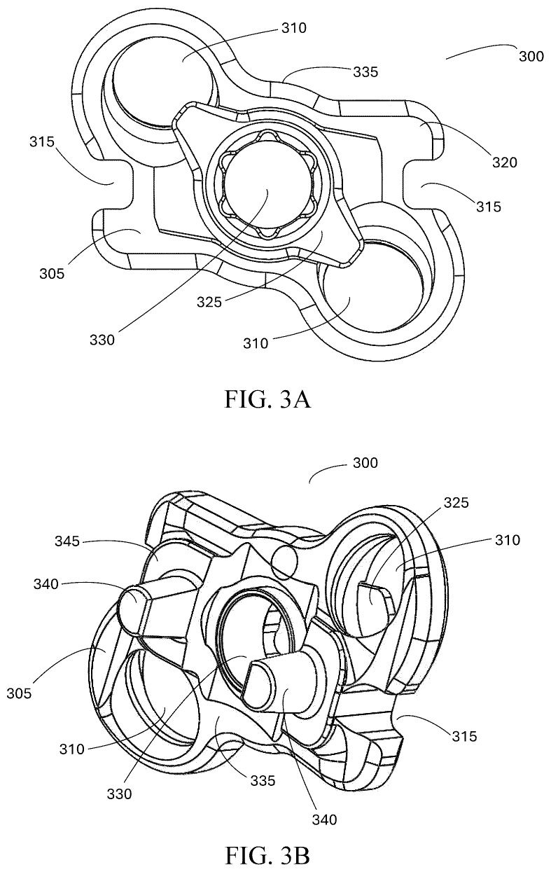

FIG. 3A illustrates another embodiment of a spinal plate 300 having a plate body 305 with an anterior surface 320, a posterior surface 335, two screw holes 310 extending from anterior surface 320 through plate body 305 to posterior surface 335. One screw hole 310 is positioned in a cephalad portion of plate body 305, and the other screw hole 310 is positioned in a caudal portion of plate body 305, the two holes 310 being positioned opposite each other relative to a through hole 330. Located between the cephalad and caudal portions of the plate body 305 are a pair of cutouts 315 on each lateral side of the plate body 305.

Plate body 305 includes a rotating locking mechanism 325 configured to allow respective bone screws to be inserted into each one of screw holes 310 when locking mechanism 325 is rotated. At the center of locking mechanism 325 is through hole 330 that extends from anterior surface 320 through plate body 305 to a posterior surface 335.

FIG. 3B is a rear or posterior perspective view of spinal plate 300 illustrating a pair of extensions 340 that extend from a shelf 345 on posterior surface 335 in a posterior direction relative to plate body 305. Each extension 340 has a frustoconical shape. Some embodiments may include only a single extension 340 though having two extensions 340 may provide greater stability between spinal plate 300 and an interbody. Through hole 330 is positioned directly between extensions 340 though such a configuration is not a requirement. The relative positioning of through hole 330 and extensions 340 is determined based on the relative positions of corresponding through holes or bores in the interbody.

FIG. 4A illustrates another embodiment of a spinal plate 400 having a plate body 405 with an anterior surface 420, a posterior surface 435, four screw holes 410 extending from anterior surface 420 through plate body 405 to posterior surface 435. Two screw holes 410 are positioned in a cephalad portion of plate body 405, and two screw holes 410 are positioned in a caudal portion of plate body 405. Located between the cephalad and caudal portions of the plate body 405 are a pair of cutouts 415 on each lateral side of the plate body 405.

Unlike the embodiment illustrated in FIGS. 1A-1D, the embodiment of FIGS. 4A and 4B includes two rotating locking mechanisms 425 each one configured to prevent the backout of two bone screws when locking mechanisms 425 are rotated. Locking mechanisms 425 are separated from each other, and positioned between them is a through hole 430 that extends from anterior surface 420 through plate body 405 to a posterior surface 435.

FIG. 4B is a rear or posterior perspective view of spinal plate 400 illustrating a pair of extensions 440 that extend from a shelf 445 on posterior surface 435 in a posterior direction relative to plate body 405. Each extension 440 has a frustoconical shape. The shape of extension 440 may be chosen to achieve a friction fit with a bore or through hole of an interbody, a friction fit that, in some embodiments, allows for a limited amount of angulation of spinal plate 400 relative to the interbody. Some embodiments may include only a single extension 440 though having two extensions 440 may provide greater stability between spinal plate 400 and an interbody. Through hole 430 is positioned directly between extensions 440 though such a configuration is not a requirement. The relative positioning of through hole 430 and extensions 440 is determined based on the relative positions of corresponding through holes or bores in the interbody.

FIG. 5A illustrates another embodiment of a spinal plate 500 having a plate body 505 with an anterior surface 520, a posterior surface 535, three screw holes 510 extending from anterior surface 520 through plate body 505 to posterior surface 535. One screw hole 510 is positioned in a cephalad portion of plate body 505, and two screw holes 510 are positioned in a caudal portion of plate body 505. Located between the cephalad and caudal portions of the plate body 505 are a pair of cutouts 515 on each lateral side of the plate body 505.

Unlike the embodiment illustrated in FIGS. 2A-2B, but similar to the embodiment of FIGS. 4A and 4B, the embodiment of FIGS. 5A and 5B includes two rotating locking mechanisms 525, the upper or cephalad locking mechanism 525 configured to prevent the backout of a cephalad bone screw and the lower or caudal locking mechanism 525 configured to prevent the backout of the two caudal bone screws when locking mechanisms 525 are rotated. Locking mechanisms 525 are separated from each other, and positioned between them is a through hole 530 that extends from anterior surface 520 through plate body 505 to a posterior surface 535.

FIG. 5B is a rear or posterior perspective view of spinal plate 500 illustrating a pair of extensions 540 that extend from a shelf 545 on posterior surface 535 in a posterior direction relative to plate body 505. Each extension 540 has a frustoconical shape. The shape of extension 540 may be chosen to achieve a friction fit with a bore or through hole of an interbody, a friction fit that, in some embodiments, allows for a limited amount of angulation of spinal plate 500 relative to the interbody. Some embodiments may include only a single extension 540 though having two extensions 540 may provide greater stability between spinal plate 500 and an interbody. Through hole 530 is positioned directly between extensions 540 though such a configuration is not a requirement. The relative positioning of through hole 530 and extensions 540 is determined based on the relative positions of corresponding through holes or bores in the interbody.

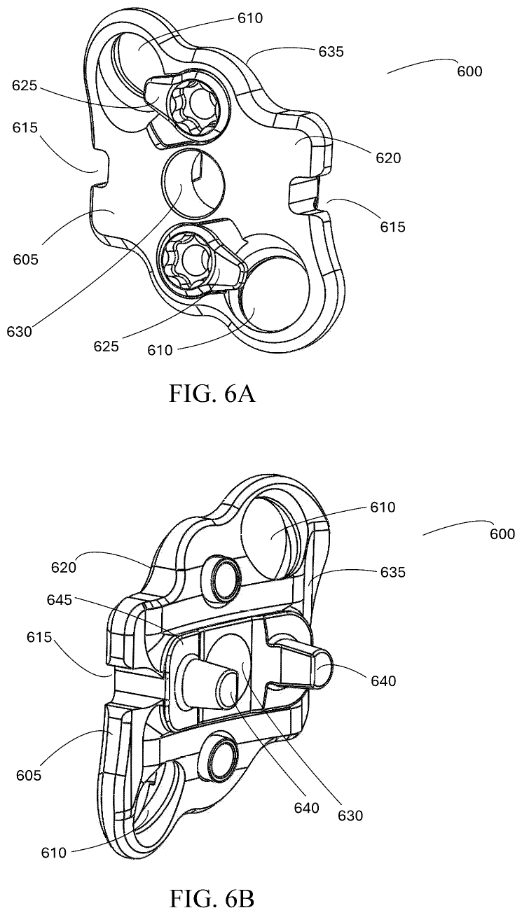

FIG. 6A illustrates another embodiment of a spinal plate 600 having a plate body 605 with an anterior surface 620, a posterior surface 635, two screw holes 610 extending from anterior surface 620 through plate body 605 to posterior surface 635. One screw hole 610 is positioned in a cephalad portion of plate body 605, and two screw holes 610 are positioned in a caudal portion of plate body 605. Located between the cephalad and caudal portions of the plate body 605 are a pair of cutouts 615 on each lateral side of the plate body 605.

Unlike the embodiment illustrated in FIGS. 3A-3B, but similar to the embodiments of FIGS. 4A and 5B, the embodiment of FIGS. 6A and 6B includes two rotating locking mechanisms 625, the upper or cephalad locking mechanism 625 configured to prevent the backout of a cephalad bone screw and the lower or caudal locking mechanism 625 configured to prevent the backout of a caudal bone screw when locking mechanisms 625 are rotated. Locking mechanisms 625 are separated from each other, and positioned between them is a through hole 630 that extends from anterior surface 620 through plate body 605 to a posterior surface 635.

FIG. 6B is a rear or posterior perspective view of spinal plate 600 illustrating a pair of extensions 640 that extend from a shelf 645 on posterior surface 635 in a posterior direction relative to plate body 605. Each extension 640 has a frustoconical shape. The shape of extension 640 may be chosen to achieve a friction fit with a bore or through hole of an interbody, a friction fit that, in some embodiments, allows for a limited amount of angulation of spinal plate 600 relative to the interbody. Some embodiments may include only a single extension 640 though having two extensions 640 may provide greater stability between spinal plate 600 and an interbody. Through hole 630 is positioned directly between extensions 640 though such a configuration is not a requirement. The relative positioning of through hole 630 and extensions 640 is determined based on the relative positions of corresponding through holes or bores in the interbody.

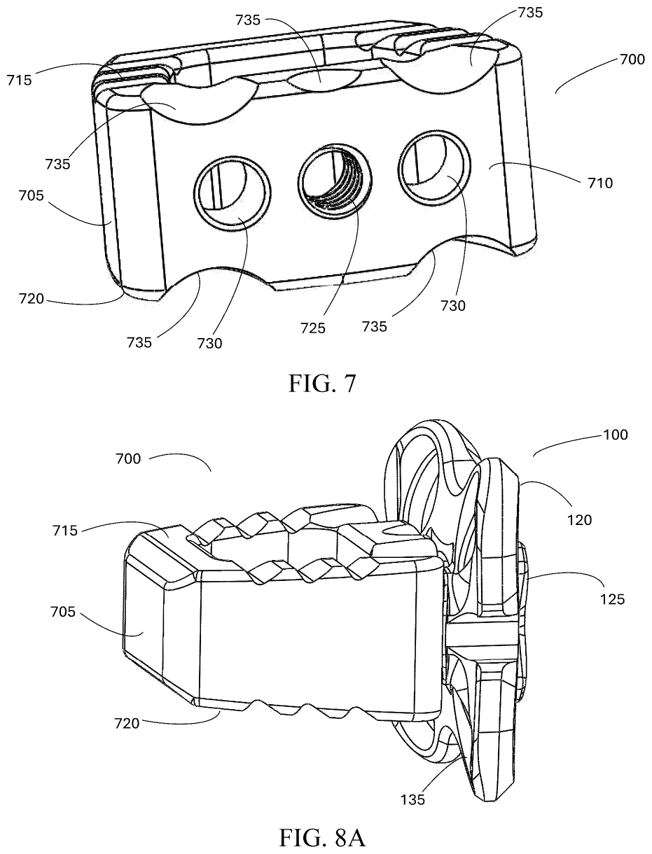

FIG. 7 illustrates an embodiment of an interbody (or spinal cage or space) 700 according to the present disclosure. Interbody 700 has a body portion 705 with an anterior face 710, a superior face 715, and an inferior face 720. Anterior face 710 includes three through holes, the center bore 725 being a threaded bore configured to engage with an inserter (not illustrated), though, for the purposes of the present disclosure, bore 725 need not be threaded. On either side of bore 725 are bores 730 that, in this embodiment, are not threaded, though they could be threaded without departing from the spirit of this disclosure. Just as bore 725 is configured to receive an inserter, bores 730 are configured to receive posterior extensions from a spinal plate, such as extensions 140 of spinal plate 100, extensions 240 of spinal plate 200, or extensions 340 of spinal plate 300. Through holes 725 and 730 extend through anterior face 710 of body portion 705, but they need not extend all the way through to achieve the goals of the present disclosure.

The respective superior and inferior portions of anterior face 710 include scalloped cutouts 735 configured to allow for bone screws to be inserted through the screw holes of a spinal plate when the plate is engaged with interbody 700. Notably, the configuration of cutouts 735 corresponds to the screw hole configurations any one of spinal plate 100, 200, or 300, such that any of these disclosed spinal plates could be used with interbody 700. The skilled person will appreciate that other plate designs consistent with the present disclosure could be used with interbody 700 and that a different interbody design could be used while remaining true to the spirit of this disclosure.

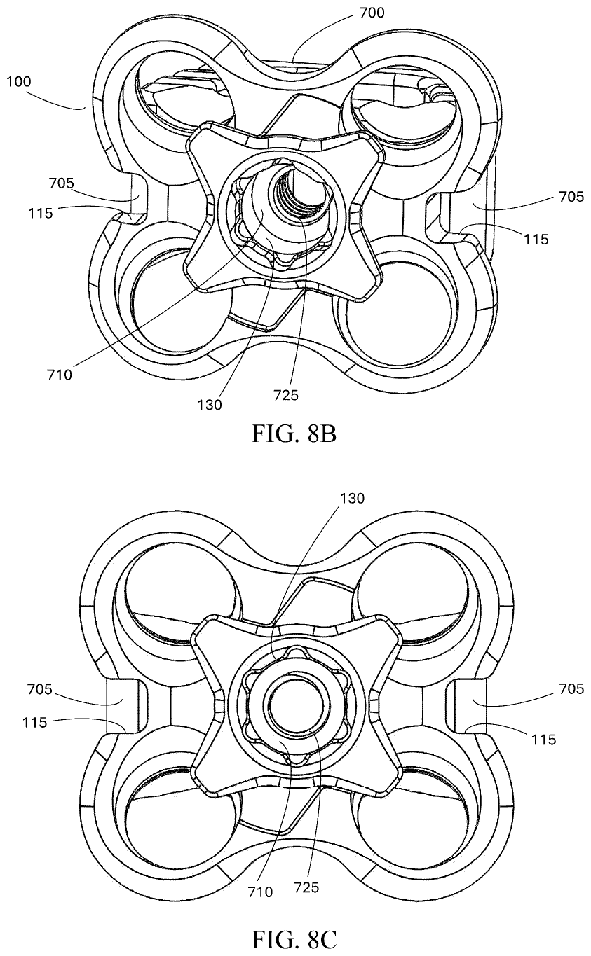

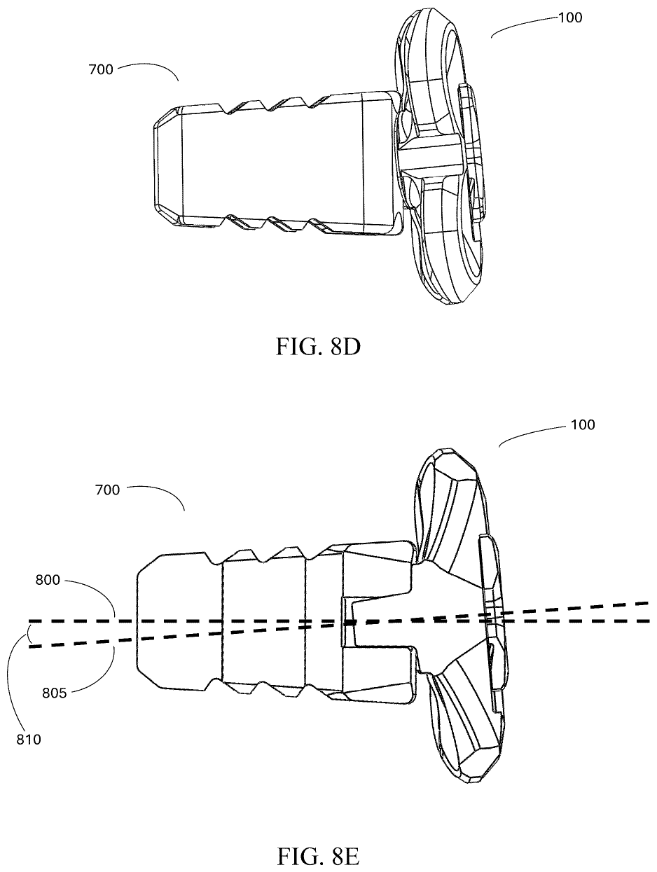

FIGS. 8A-8E illustrate spinal plate 100 press fit into (or connected to) interbody 700. FIG. 8A is a side perspective view of spinal plate 100 and interbody 700 illustrating how extensions 140 press fit into bores 730 align spinal plate 100 relative to interbody 700. This alignment is further illustrated in FIG. 8B in which bore 725 of interbody 700 is axially aligned with central hole 130 of spinal plate 100. Nevertheless, the ability to access bore 725 through central hole 130 allows an inserter to be attached to interbody 700 and, thereby, hold in place spinal plate 100 while the two elements are advanced toward a surgical site and interbody 700 is positioned in an intervertebral disc space. Although axial alignment may facilitate the goals of the present disclosure, such alignment is not a requirement.

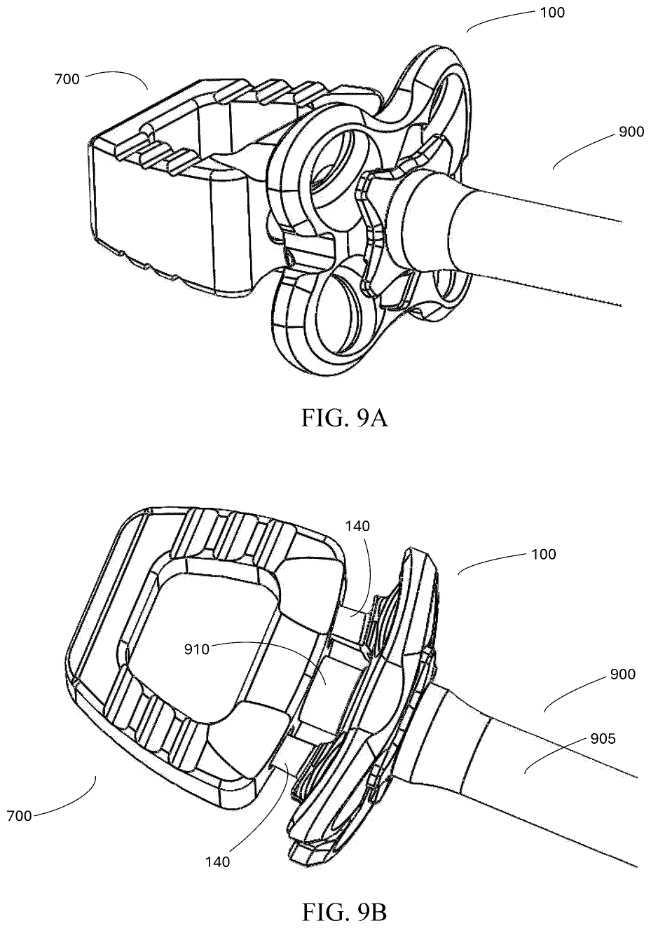

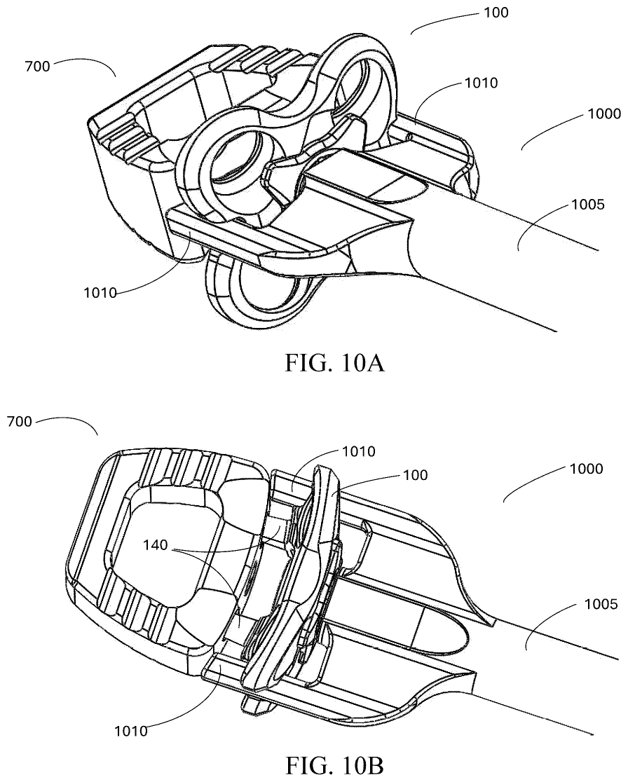

FIGS. 8B and 8C illustrate that a portion of body portion 705 of interbody 700 is visible through cutouts 115 of spinal plate 100. Moreover, a portion of anterior face 710 of body portion 705 is visible through central hole 130 because the inner diameter of central hole 130 is larger than the inner diameter of bore 725. These two ways of seeing and accessing interbody 700 allow a user to adjust the position of interbody 700 in an intervertebral disc space without having to remove spinal plate 100, which may be secured to respective vertebral bodies prior to any such adjustment of interbody 700. Such access may be achieved using a tamp as illustrated in FIGS. 9A-10B.

FIGS. 9A and 9B illustrate the use of a tamp 900 that accesses interbody 700 via central hole 130. Such access may be desirable to advance interbody 700 further into the disc space. Specifically, tamp 900 includes an elongate portion 905 with a distal extension 905 that is sized to fit through central hole 130 of tamp 900. FIG. 9B illustrates the use of tamp 900 to advance interbody 700 distally or posteriorly from spinal plate 100 introducing some space between spinal plate 100 and interbody 700 though with extensions 140 still at least partially extending within bores 730. Though according to some methods of the present disclosure, extensions 140 need not stay within bores 730 particularly if one or more screws have been advanced through one or more of screw holes 110 to secure spinal plate 100 to one or both of the underlying vertebral bodies.

FIGS. 10A and 10B illustrate the use of a tamp 1000 that accesses interbody 700 via cutouts 115. Tamp 1000 includes an elongate portion 1005 with a pair of distal prongs 1010 that are sized and positioned to align with cutouts 115 to access interbody 1000. Again, such access may be desirable to advance interbody 1000 further into the disc space, though adjustments may also be made to the angle of interbody 1000 relative to spinal plate 100 particularly by pressing against only one side interbody 1000 and not the other. Although not illustrated, such adjustments could be made with a tamp that accesses interbody 1000 through only one cutout 115. FIG. 10B illustrates the use of tamp 1000 to advance interbody 1000 distally or posteriorly from spinal plate 100 introducing some space between spinal plate 100 and interbody 1000 though with extensions 140 still at least partially extending within bores 730.

Returning to FIGS. 8A-8E that illustrate spinal plate 100 press fit into interbody 700, FIGS. 8D and 8E illustrate the ability of spinal plate 100 to pivot relative to interbody 700 by virtue of the design of extensions 100. FIG. 8E is a cross-sectional view that illustrates an imaginary line 800 bisecting interbody 700 and an imaginary line 805 bisecting spinal plate 100. Angle 810 is the angle that can be achieved between imaginary lines 800 and 805. In some embodiments, the maximum value of angle 810 is any value from about 1 degree to about 20 degrees, from about 2 degrees to about 15 degrees, from about 3 degrees to about 10 degrees, or from about 4 degrees to about 6 degrees. In some embodiments, the maximum value of angle 510 is at least about 2 degrees, at least about 5 degrees, at least about 8 degrees, at least about 11 degrees, at least about 14 degrees, or at least about 17 degrees.

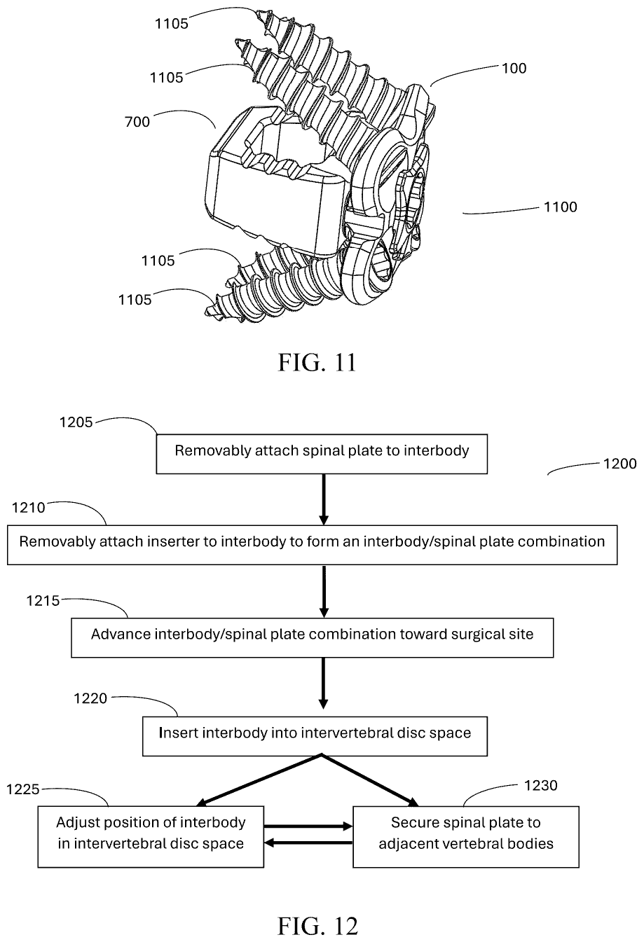

FIG. 11 illustrates an embodiment of a system 1100 that includes spinal plate 100, interbody 700, and a plurality of bone screws 1105. Each one of the plurality of bone screws 1105 is inserted into a respective screw hole 110 of spinal plate 100. The pair of bone screws 1105 in the cephalad screw holes 110 are angled in a cephalad or superior direction so as to find purchase in a first vertebral body that is above or superior to interbody 700. The pair of bone screws 805 in the caudal screw holes 110 are angled in a caudal or inferior direction so as to find purchase in a second vertebral body that is below, caudal, or inferior to interbody 700. The exact angle of each bone screw 1105 can be adjusted by a user as bone screw 1105 is advanced through screw hole 110 into the first and second vertebral bodies.

FIG. 12 is a process flow diagram illustrating a method 1200 according to the present disclosure for using the spinal plates, interbodies, inserters, and tamps as described herein.

Method 1200 begins with the step 1205, which is when a spinal plate as disclosed herein is removably attached to an interbody as disclosed herein. The spinal plate is removably attached to the interbody by inserting the projection(s) on the posterior side of the spinal plate into the bore(s) in the anterior face of the interbody. When press fit together, the spinal plate may be able to pivot relative to the interbody without becoming detached, and a certain amount of force may be required to separate the spinal plate from the interbody.

Method 1200 proceeds to step 1210, which is the attachment of an interbody inserter to the interbody. Attaching the inserter to the interbody involves inserting a distal tip or engagement mechanism of the inserter through a central hole or bore in the interbody and into a bore in the anterior face of the interbody, which bore may be threaded or otherwise configured to releasably engage the distal tip or engagement mechanism of the inserter. With the spinal plate already press fit with the interbody, the attachment of the inserter to the interbody sandwiches the spinal plate between the inserter and the interbody so that the two may be manipulated as one.

Method 1200 proceeds to step 1215, which is the advancement of the interbody/spinal plate combination toward a surgical site with the use of the inserter. The surgical site will typically be an intervertebral disc space with a damaged or diseased disc. The interbody/spinal plate combination may be advanced to the surgical site through a retractor or other device configured to create a surgical corridor for accessing the surgical site.

Method 1200 proceeds to step 1220, which is the insertion of the interbody into the intervertebral disc space. Inserting the interbody into the disc space places the spinal plate against the vertebral bodies above and below the disc space. Because the spinal plate is pivotable relative to the interbody, positioning the interbody in the disc space may cause the spinal plate to pivot in response to the shapes and/or relative positions of the vertebral bodies.

Method 1200 may proceed to an interbody adjustment step 1225 or a spinal plate securing step 1230. If step 1225 follows step 1220, then step 1230 will follow step 1225. In some embodiments, step 1230 is performed between steps 1220 and 1225.

Interbody adjustment step 1225 may involve the use of a tamp that is configured to reach through or around the spinal plate to contact the interbody. In some embodiments, it may be desirable to position the interbody further into the disc space and/or to adjust the angle of the interbody within the disc space. The central hole or bore and/or cutouts on the side of the spinal plate are configured to accommodate this adjustment action of the tamp. In some embodiments, as the position of the interbody is adjusted in the disc space with the spinal plate remaining stationary, the posterior extension(s) on the spinal plate may remain at least partially within one or more bores of the interbody as illustrated in FIGS. 9A and 10A.

Spinal plate securing step 1230 involves the advancement of a plurality of bone screws through the screw holes in the spinal plate and further advancing the bone screws into the respective vertebral bodies so as to fix the position of the spinal plate. As indicated above, it is possible to adjust the position of the interbody after the spinal plate has been secured by use of a tamp, such as illustrated in FIGS. 9B and 10B.

### EMBODIMENTS

The following embodiments are provided as examples only of specific configurations, materials, arrangements, etc. contemplated by the authors of this disclosure:

Embodiment 1. A spinal plate comprising:
a plate body having a thickness between an anterior surface and a posterior surface;
a plurality of screw holes extending from the anterior surface to the posterior surface; and
a projection extending from the posterior surface, the projection positioned and sized so as to be releasably received by a bore in an interbody; and
wherein the projection allows the plate body to pivot relative to the interbody while the projection is received into the bore.

Embodiment 2. The spinal plate of Embodiment 1, wherein the projection defines a frustoconical shape.

Embodiment 3. The spinal plate of either Embodiment 1 or 2, wherein the projection is a frustoconical cone having an angle that is between about 2 degrees and about 12 degrees, between about 5 degrees and about 15 degrees, between about 8 degrees and about 18 degrees, between about 11 degrees and about 21 degrees, or between about 14 degrees and about 24.

Embodiment 4. The spinal plate of any one of Embodiments 1 through 3, wherein the projection is configured to be press fit into the bore the interbody so as to require a certain force to remove the projection from the bore.

Embodiment 5. The spinal plate of any one of Embodiments 1 through 4, wherein the plate body further comprises a first lateral cutout and, optionally, a second lateral cutout that allows access to at least an edge of the interbody.

Embodiment 6. The spinal plate of any one of Embodiments 1 through 5, wherein the plate body further comprises a central bore configured to allow access through the plate to the interbody.

Embodiment 7. The spinal plate of Embodiment 6, wherein the central bore is positioned so as to align with a bore of the interbody so that an interbody inserter engaged to the interbody can extend through the central bore of the plate to secure the plate during insertion of the interbody into an intervertebral disc space.

Embodiment 8. The spinal plate of any one of Embodiments 1 through 7, wherein the plurality of screw holes consists of four screw holes.

Embodiment 9. The spinal plate of any one of Embodiments 1 through 7, wherein the plurality of screw holes consists of three screw holes.

Embodiment 10. The spinal plate of any one of Embodiments 1 through 7, wherein the plurality of screw holes consists of two screw holes.

Embodiment 11. The spinal plate of any one of Embodiments 1 through 8, further comprising a second projection extending from the posterior surface, the second projection positioned and sized so as to be releasably received by a second bore in the interbody

Embodiment 12. The spinal plate of Embodiment 11, wherein the projection and the second projection are positioned opposite each other.

Embodiment 13. The spinal plate of any one of Embodiments 1 through 12, further comprising a shelf located on the posterior surface, wherein the projection(s) extend posteriorly from the shelf.

Embodiment 14. A system comprising:
a spinal plate of any one of Embodiments 1 through 13;
an interbody having an anterior face and a bore therein sized to receive the projection of the spinal plate and, optionally, a second bore sized to receive the second projection of the spinal plate; and
a plurality of bone screws each one configured to be advanced through a respective one of the plurality of screw holes of the spinal plate.

Embodiment 15. The system of Embodiment 14, wherein the shelf, the projection, and, optionally, the second projection, is/are sized so as to position the anterior face of the interbody proud relative to an anterior surface of a vertebral body adjacent to which the interbody is configured for implantation.

Embodiment 16. The system of Embodiment 14, wherein the shelf, the projection, and, optionally, the second projection, is/are sized so as to position the anterior face of the interbody flush with an anterior surface of a vertebral body adjacent to which the interbody is configured for implantation.

Embodiment 17. The system of Embodiment 14, wherein the shelf, the projection, and, optionally, the second projection, is/are sized so as to position the anterior face of the interbody sub-flush relative to an anterior surface of a vertebral body adjacent to which the interbody is configured for implantation.

Embodiment 18. The system of any one of Embodiments 14 through 17, further comprising a tamp configured to apply a force selectively to the interbody that is positioned posteriorly to the spinal plate when the interbody is positioned in an intervertebral disc space.

Embodiment 19. The system of Embodiment 18, wherein the tamp comprises a pair of lateral prongs configured to pass through the first lateral cutout and, optionally, the second lateral cutout of the spinal plate to contact the interbody that is positioned posteriorly to the spinal plate when the interbody is positioned in an intervertebral disc space.

Embodiment 20. The system of Embodiment 18, wherein the tamp comprises a central extension configured pass through the spinal plate to contact the interbody that is positioned posteriorly to the spinal plate when the interbody is positioned in an intervertebral disc space.

Embodiment 21. The system of any one Embodiments 14 through 20, further comprising an inserter having a distal portion and a proximal portion, the distal portion configured to engage a central bore of the interbody with the spinal plate positioned between the interbody and the proximal portion of the inserter.

Embodiment 22. The system of any of Embodiments 14 through 21, wherein a maximum angle between an imaginary line bisecting the interbody and an imaginary line bisecting the spinal plate is at least about 2 degrees, at least about 4 degrees, at least about 6 degrees, or at least about 8 degrees.

Embodiment 23. A method of implanting the system of any one of Embodiments 14 through 22, the method comprising:
press fitting the spinal plate to the interbody to form a spinal plate/interbody combination, wherein press fitting the spinal plate to the interbody comprises inserting the projection into the bore, which, when press fit together, allows the spinal plate to pivot relative to the interbody;
securing a distal end of an inserter to the interbody thereby holding the spinal plate between the interbody and the inserter;
advancing the spinal plate/interbody combination toward a surgical site;
inserting the interbody into an intervertebral disc space; and
securing the spinal plate to a vertebral body cranial to the intervertebral disc space and to a vertebral body caudal to the intervertebral disc space by advancing each one of the plurality of bone screws through the plurality of screw holes, respectively.

Embodiment 24. The method of Embodiment 23, further comprising:
after inserting the interbody into the intervertebral disc space, detaching and removing the inserter from the interbody; and
adjusting the position of the interbody in the intervertebral disc space independent of the position of the spinal plate.

Embodiment 25. The method of Embodiment 24, wherein the adjusting the position of the interbody independent of the position of the spinal plate is achieved using a tamp that reaches past the spinal plate to contact the interbody.

Embodiment 26. The method of Embodiment 24, wherein the adjusting the position of the interbody independent of the position of the spinal plate is achieved using a tamp that reaches through the spinal plate to contact the interbody.

The method of any one of Embodiments 23 through 26, wherein the projection is maintained in the bore as the position of interbody is adjusted.

The method of any one of Embodiments 23 through 27, further comprising advancing a plurality of bone screws, one through each of the plurality of screw holes, into the respective vertebral bodies.

The method of Embodiment 28, wherein the interbody is adjustable within the intervertebral disc space irrespective of whether the bone screws have been secured to the respective vertebral bodies.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular approach (e.g., anterior); however, other approaches (e.g., lateral, posterior, transforaminal, etc.) are also contemplated.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. In one embodiment, the terms “about” and “approximately” refer to numerical parameters within 10% of the indicated range.

The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Furthermore, if any references have been made to patents and printed publications throughout this disclosure, each of these references and printed publications are individually incorporated herein by reference in their entirety.

In closing, it is to be understood that the embodiments disclosed herein are illustrative of the principles of the present disclosure. Other modifications that may be employed are within the scope of this disclosure. Thus, by way of example, but not of limitation, alternative configurations of the embodiments of the present disclosure may be utilized in accordance with the teachings herein. Accordingly, the present disclosure is not limited to that precisely as shown and described.

1. A spinal plate system comprising:
an interbody having an anterior surface, at least bore and at least one screw hole in the anterior surface;
a plate body having a thickness between an anterior surface and a posterior surface;
a plurality of screw holes extending from the anterior surface to the posterior surface; and
a projection extending from the posterior surface, the projection positioned and sized so as to be releasably received by the bore in the interbody;
wherein the projection is unthreaded and configured to be press-fit into the bore of the interbody so as to require a force to remove the projection from the bore;
wherein the projection allows the plate body to pivot relative to the interbody while the projection remains press-fit in the bore.

2. The spinal plate of claim 1, wherein the projection defines a frustoconical shape.

3. The spinal plate of claim 1, wherein the projection is defined by one or more side walls having an angle relative to the plate body that is between about 2 degrees and about 15 degrees.

4. (canceled)

5. The spinal plate of claim 1, wherein the plate body further comprises at least one lateral cutout that allows access to at least an edge of the interbody.

6. The spinal plate of claim 1, wherein the plate body further comprises a central bore configured to allow access through the plate to the interbody.

7. The spinal plate of claim 6, wherein the central bore is positioned so as to align with a bore of the interbody so that an interbody inserter engaged to the interbody can extend through the central bore of the plate to secure the plate during insertion of the interbody into an intervertebral disc space.

8. The spinal plate of claim 1, wherein the plurality of screw holes consists of four screw holes.

9. The spinal plate of claim 1, wherein the plurality of screw holes consists of three screw holes.

10. The spinal plate of claim 1, wherein the plurality of screw holes consists of two screw holes.

11. The spinal plate of claim 1, further comprising a second projection extending from the posterior surface, the second projection positioned and sized so as to be releasably received by a second bore in the interbody.

12. The spinal plate of claim 11, wherein the projection and the second projection are positioned opposite each other.

13. The spinal plate system of claim 1, further comprising:
a plurality of bone screws each one configured to be advanced through a respective one of the plurality of screw holes of the spinal plate.

14. The system of claim 13, wherein the projection or a shelf located on the posterior surface of the spinal plate is sized so as to position the anterior face of the interbody flush with an anterior surface of a vertebral body adjacent to which the interbody is configured for implantation.

15. The system of claim 13, wherein the projection or a shelf located on the posterior surface of the spinal plate is sized so as to position the anterior face of the interbody sub-flush relative to an anterior surface of a vertebral body adjacent to which the interbody is configured for implantation.

16. The system of claim 13, further comprising a tamp configured to apply a force selectively to the interbody that is positioned posteriorly to the spinal plate when the interbody is positioned in an intervertebral disc space.

17. The system of claim 16, wherein the tamp comprises a pair of lateral prongs configured pass through at least one lateral cutout of the spinal plate to contact the interbody that is positioned posteriorly to the spinal plate when the interbody is positioned in the intervertebral disc space.

18. The system of claim 16, wherein the tamp comprises a central extension configured pass through the spinal plate to contact the interbody that is positioned posteriorly to the spinal plate when the interbody is positioned in the intervertebral disc space.

19. The system of claim 13, further comprising an inserter having a distal portion and a proximal portion, the distal portion configured to engage a central bore of the interbody with the spinal plate positioned between the interbody and the proximal portion of the inserter.

20. The system of claim 1, wherein a maximum angle between an imaginary line bisecting the interbody and an imaginary line bisecting the spinal plate is at least about 4 degrees.
